(19) United States
(12) Patent Application Publication
(10) Pub. No.: US 2015/0147418 A1
(43) Pub. Date: May 28, 2015

(54) SLEEP QUALITY IMPROVING AGENT

(71) Applicant: LION CORPORATION, Sumida-ku, Tokyo (JP)

(72) Inventors: Yoshihiro Urade, Ibaraki (JP); Ikuko Kamada, Tokyo (JP); Toshio Gokita, Tokyo (JP); Kiwamu Suzuki, Tokyo (JP); Hiroaki Kambayashi, Tokyo (JP); Michiaki Murakoshi, Tokyo (JP)

(73) Assignee: LION CORPORATION, Sumida-ku, Tokyo (JP)

(21) Appl. No.: 14/402,245

(22) PCT Filed: Jun. 26, 2013
(86) PCT No.: PCT/JP2013/067530

§ 371 Date: Nov. 19, 2014

(30) Foreign Application Priority Data
Jul. 25, 2012 (JP) 2012-164926

(51) Int. Cl. A61K 36/87; A61K 31/7048; A61K 31/05; A61K 36/63; A61K 36/17

(52) U.S. Cl. CPC A61K 36/87; A61K 36/63; A61K 36/17; A61K 31/05; A61K 31/7048
USPC 424/766; 424/777; 424/769

(57) ABSTRACT

An object of the present invention is to provide a sleep quality improving agent which has a sufficient sleep quality improving effect, can exert makes the effect even through oral administration in any dosage form, and has ensured safety to the body. That is, the present invention provides: a sleep quality improving agent which contains a stilbene-based compound and/or an olive extract as an active ingredient, and a sleep quality improving composition which contains the sleep quality improving agent.

### TECHNICAL FIELD

The present invention relates to a sleep quality improving agent.

### BACKGROUND

Sleep is roughly divided into two types: non-REM sleep, during which the majority of the cerebrum activity is suspended; and REM sleep, during which a part of brain operates actively with a dream action, while the whole body is under condition of draining of strength. These two types of sleep are repeated at a certain interval, and thereby form sleep (FIG. 1). A good sleep quality requires that the time from the onset of sleep to the appearance of the non-REM sleep (sleep onset latency) be short, and requires that enough time of the non-REM sleep as well as deep non-REM sleep, especially enough deep non-REM sleep in the initial stage of sleep, be secured.

However, as the 24-hour society proceeds in recent years, the average sleep time of Japanese is significantly reduced, and simultaneously the number of patients complaining of sleep is increasing. According to the attitude survey of health promotion in Heisei 8 (1996), it is reported that 21.4% of people are complaining of sleep for some reasons. Also, focusing on changes in sleep by increasing age, it is reported that the time of the non-REM sleep is significantly reduced, and the quality of sleep is deteriorating.

The complaints about sleep include having a difficulty in falling asleep, having a nightmare, feeling sleepy on awakening in the morning, not feeling like having a sound sleep, feeling fatigue on awakening in the morning, feeling sleepy during daytime, and the like. These may lead to lowering an efficiency of work, or an unexpected accident. The causes for those include not only a short sleep time, but also lack of having a good quality sleep. That is, the time from the onset of sleep to the appearance of the non-REM sleep (sleep onset latency) is too long, the time of the non-REM sleep is too short, or the deep non-REM sleep is not obtained. There may be an option to use pharmaceutical sleeping pills to treat them. However, taking the sleeping pills requires a doctor's diagnosis, and there are side effects such as uncomfortable feelings on awakening, memory disorder, and dependency, thus the pill may not be used easily and safely. Furthermore, there are some pharmaceutical products such as benzodiazepine-based sleeping pills and barbiturate-based sleeping pills, which reduce the non-REM sleep and may actually lower the quality of sleep if not used properly.

Apart from the pharmaceutical agents, research and development on sleep improving agents made from natural ingredients and foods and drinks have been vigorously conducted, and thereby various such agents have been proposed. As active ingredients for such sleep improving agents, for example, ingredients derived from a plant of the genus Withania of the family Solanaceae (Patent Literature 1), fermentation products of Hemerocallis fulva var. semipervirens (Patent Literature 2), theanine derived from tea leaves (Patent Literature 3), Korean ginseng extracts (Non Patent Literature 1), and the like have been proposed. However, the effects of these ingredients on the sleep onset latency, the time of the non-REM sleep, and the depth of the non-REM sleep remain unclear, and their improving action on the quality of sleep is not sufficient.

### RELATED ART DOCUMENTS

### Patent Literature

Patent Literature 1: Japanese Patent Application Laid-open No. 2006-28051
Patent Literature 2: Japanese Patent Application Laid-open No. 2006-62998
Patent Literature 3: International Publication No. 2005/097101

### Non Patent Literature

Non Patent Literature 1: Young Ho Rhee et al., Psychopharmacology, 101, 486-488 (1990)

### SUMMARY OF THE INVENTION

### Problem to be Solved by the Invention

The present invention has been made in view of the above, and aims to provide a sleep quality improving agent that has a satisfactory sleep quality improving effect, that can exert the effect even through oral administration in any dosage form, and that has ensured safety to the body.

### Solution to Problem

The present inventors carried out intensive studies to achieve the goal described above. As a result, it has been found that a stilbene-based compound and/or an olive extract have an effect on improving the quality of sleep, thus achieving the present invention.

The present invention provides the following inventions.

[1] A sleep quality improving agent comprising a stilbene-based compound and/or an olive extract as an active ingredient.
[2] The sleep quality improving agent according to [1], wherein the stilbene-based compound is a plant extract comprising the stilbene-based compound.
[3] The sleep quality improving agent according to [2], wherein the plant extract comprising the stilbene-based compound is a Vitaceae plant extract comprising the stilbene-based compound, or a Gnetaceae plant extract comprising the stilbene-based compound.
[4] The sleep quality improving agent according to any one of [1] to [3], wherein the olive extract is an extract isolated from a fruit of olive.
[5] The sleep quality improving agent according to any one of [1] to [4], wherein the olive extract is an olive extract comprising one or more polyphenols selected from the group consisting of hydroxytyrosol, tyrosol, and oleuropein.
[6] The sleep quality improving agent according to any one of [1] to [5], for increasing a ratio of non-REM sleep time to a whole sleep time.
[7] The sleep quality improving agent according to any one of [1] to [6], for deepening a non-REM sleep at an initial stage of sleep.
[8] The sleep quality improving agent according to any one of [1] to [7], for improving less sleepiness feeling on awakening.
[9] The sleep quality improving agent according to any one of [1] to [8], for improving sleep initiation and soundness of sleep.
[10] The sleep quality improving agent according to any one of [1] to [9], for improving dreaming.
[11] The sleep quality improving agent according to any one of [1] to [10], for improving feeling of fatigue.
[12] The sleep quality improving agent according to any one of [1] to [11], for improving satisfaction with a length of sleep time.
[13] The sleep quality improving agent according to any one of [1] to [12], which is an oral agent.
[14] A sleep quality improving composition comprising the sleep quality improving agent according to any one of [1] to [13].
[15] The sleep quality improving composition according to [14], which is an oral agent.
[16] A method for utilizing a stilbene-based compound and/or an olive extract in order to improve quality of sleep.

Furthermore, the present invention can provide the following embodiments in other aspects.

[17] A method for improving a quality of sleep, or a method for inducing natural sleep, comprising the step of administering a stilbene-based compound and/or an olive extract.
[18] The method described in [17] above, wherein the stilbene-based compound and/or the olive extract are administered orally.
[19] Use of a stilbene-based compound and/or an olive extract for manufacturing a sleep quality improving agent.

### Effect of the Invention

According to the present invention, a sleep quality improving agent is provided, which has a satisfactory sleep quality improving effect, can exert the effect even through oral administration in any dosage form, and has ensured safety to the body.

### BRIEF DESCRIPTION OF DRAWINGS

FIG. 1 is a diagram showing a general pattern of sleeping.

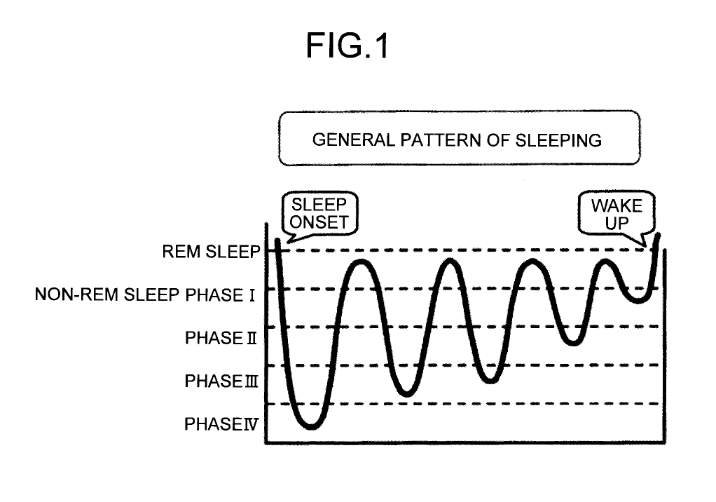

### EMBODIMENTS FOR CARRYING OUT THE INVENTION

In the present invention, a stilbene-based compound and/or an olive extract are comprised as an active ingredient.

### (1) Stilbene-Based Compound

A stilbene-based compound is a compound having a stilbene structure. The stilbene structure is represented by the formula (I).

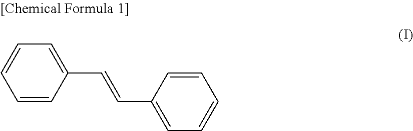 [SMILES: C(=C\c1ccccc1)/c1ccccc1]

The stilbene structure may be substituted by a functional group. The substitution site by the functional group is not particularly limited, and they are usually an aromatic ring. Examples of such functional groups may include a hydroxyl group, an alkyl group, a hydroxyalkyl group, an amino group, a sulfone group, and a phosphate group. Among them, a hydroxyl group and a hydroxyalkyl group are preferable.

The stilbene-based compound may be a monomer, a polymer, a monomeric glycoside, or a polymeric glycoside as long as the compound has a stilbene structure. Among them, a monomer, a polymer, and glycosides thereof wherein the functional group consists of a hydroxyl group and/or a hydroxyalkyl group are preferable. Examples of the monomer may include Resveratrol, Piceatannol, Astringin, and Pterostilbene. Examples of the monomeric glycoside may include Piceid. Examples of the polymer may include δ-viniferin, ε-viniferin, Gnetin C, Gnetin L, α-viniferin, and Ampelopsin A. Examples of the polymeric glycoside may include Gnemonoside A, Gnemonoside C, and Gnemonoside D.

The above mentioned compounds are classified as resveratrols. The resveratrols are known to have anti-ischemic heart disease effect (Lancet, 338, 1523-1526, 1992), anti-cancer effect (Br. J. cancer, 86, 774-778, 2002), reduction effect of onset of dementia (Am. J. Epidemiol, 167, 648-691, 2008), inhibitory effect of hypertension (Eur. J. Pharmacol., 667, 258-264, 2011), and effect of life extension (Nature, 444, 337-342, 2006). The stilbene-based compound is preferably one or more compounds selected from the resveratrols.

In the present invention, the stilbene-based compound may be a stilbene-based compound obtained by chemical synthesis, or a naturally derived stilbene-based compound (for example, derived from bacterial cells, or plants). Among them, the naturally derived stilbene-based compound is preferable, and the stilbene-based compound derived from the plant is preferable.

The naturally derived stilbene-based compound does not need to be a pure stilbene-based compound and may be a mixture with other compounds. Examples of the mixture may include a plant extract, a plant body in part or in whole, and a culture of bacteria cells. Among them, a plant extract is preferable.

When a plant extract is used as the stilbene-based compound, a plant extract extracted from a plant comprising the stilbene-based compound may be used. Examples of the plants comprising the stilbene-based compound may include a Vitaceae plant, a Dipterocarpaceae plant, a Leguminous plant, a Cyperaceae plant, and a Gnetaceae plant. Among them, a Vitaceae plant or a Gnetaceae plant is preferable.

A Vitaceae plant may be used so long as the plant comprises the stilbene-based compound, and preferably contains the resveratrol(s). Examples of species of the Vitaceae plant may include Vitis vinifera, Vitis labrusca, Vitis california, Vitis amurensis, Vitis coignetiae, and Vitis shiragai. Examples of cultivars of the Vitaceae plant may include Cabernet Sauvignon, Merlot, Pinot Noir, Gamay, Syrah, Nebbiolo, Sangiovese, Tempranillo, Zinfandel, Carmenere, Malbec, Muscat Bailey A, Koshu, Sauvignon Blanc, Sémillon, Chardonnay, Muscadet, Riesling, Musca, Viognier, Trebbiano, Pinot Gridio, Furmint, and Palomino.

A species of the Gnetaceae plant are not particularly limited so long as they comprise the resveratrol(s). Examples of the Gnetaceae plants may include Gnetum gnemon (melinjo), Gnetum brunonianum, Gnetum latifolium, and Gnetum tenuifolium. Among them, Gnetum gnemon is preferable.

An extraction part of the plant is not particularly limited, and they may be the whole of the plant body or a part thereof body such as leaf, stem, vine, leaf, fruit, and seed. In case of the Vitaceae plant, a stem, a vine, a leaf, or a fruit including peel and seed is preferable. In case of the Gnetaceae plant, a fruit, a seed, an endosperm of the fruit, a flower, or a leaf is preferable, the fruit, the seed, or the endosperm of the fruit is more preferable, and the endosperm of the fruit is further preferable.

When a part of the plant body is used, the relevant part isolated from the plant body may be used as is, or after drying by the sun, a machine, or the like.

A method of extraction is not particularly limited, but examples of the methods may include a solvent extraction method and a supercritical extraction method using carbon dioxide and the like. In the solvent extraction method, examples of the extracting solvent may include water, methanol, ethanol, n-propanol, isopropanol, acetone, methyl ethyl ketone, methyl butyl ketone, ethyl acetate, ethylene glycol, propylene glycol, glycerin, acetic acid, and propionic acid. These extracting solvents may be used alone or as a mixture in any combination of two or more. Preferably, the extracting solvent is water, ethanol or a mixture thereof (water-containing ethanol). In case that the water-containing ethanol is used, although the concentration of alcohol is not limited, it is preferably 20 to 90 volume %, more preferably, 40 to 80 volume %.

Here, the plant extract may be used, or the stilbene-based compound obtained by purifying the above plant extract may be used.

A grape extract and a melinjo extract are commercially available, and the commercially available products may be used in the present invention. Examples of commercially available grape extracts may include “VINEATROL20M (trade name)” (manufactured by ACTICHEM S.A.). Examples of commercially available melinjo extracts may include “MelinjoResveratrol-20 (trade name)” (manufactured by Luna YBF Inc.).

When the sleep quality improving agent of the present invention uses the stilbene-based compound as an active ingredient, the dosage of the sleep quality improving agent is not particularly limited so long as the effect of the present invention is not adversely affected, and may be suitably adjusted according to factors such as the age and the condition of the subject to be administered. The dosage per day for the administered subject to obtain the intended effect is generally 0.1 to 1000 mg, preferably 1 to 500 mg, more preferably 10 to 200 mg, and yet more preferably 20 to 120 mg as an amount of ingestion of the stilbene-based compound per day.

(2) Olive Extract

An olive extract is an extract isolated from the whole or a part of plant body of olive (Olea europaea L.). The extract may be isolated from the whole or a part of the olive plant body by using an extraction solvent or by squeezing.

An isolation part for the olive extract is not particularly limited, and may be the whole plant body or a part thereof such as leaf, flower, and fruit, but the fruit is preferable.

As the extraction solvent, for example, water, an organic solvent (for example, a single solvent or a mixed solvent of two or more selected from alcohol, acetone and the like), or a mixed solution of water and an organic solvent may be used. Among them, water, a mixed liquid of water and alcohol, and alcohol are preferable. Ethanol and methanol are preferable as the alcohol. An extraction time and temperature may be suitably determined according to the extraction part for the olive extract and the type of the solvent. The olive extract may be a crude extract extracted from olive, or may be obtained from a crude extract through the process such as concentration, drying, and pulverization. Further, the extract may be used after removing impurities by treatment using a partition method and a purification treatment (for example, an absorptive treatment using an ion exchange resin, a column and the like, subsequent elution with a solvent followed by further concentration if necessary).

When the olive extract is isolated by squeezing, an isolation object is preferably a fruit, and more preferably a fully ripened olive fruit. For example, the olive extract may be obtained by filtrating and/or concentrating a fruit juice that was obtained by squeezing the fruit. These filtrate and concentrate may be optionally centrifuged or pulverized by spray drying and the like. A content of polyphenols such as hydroxytyrosol, tyrosol and oleuropein in the olive extract may be increased by carrying out centrifugation.

A form of the olive extract is not particularly limited, and may be powder or paste.

Olive is generally rich in glycerides of fatty acids such as a glyceride of oleic acid, a glyceride of linoleic acid, and a glyceride of palmitic acid, and fat-soluble vitamins, and contains minerals such as potassium, phosphorus, and magnesium. Olive oil contains fat-soluble ingredients such as unsaturated fatty acids, (3-carotene, vitamin D and E, and squalene dihydroquercetin, and polyphenols such as hydroxytyrosol, tyrosol, and oleuropein. In the present invention, it is preferable that pulp and oil contents originally existing in olive be removed from the olive extract by an operation such as squeeze and concentration (centrifugation and the like).

In the present invention, the olive extract preferably comprises polyphenols, and more preferably comprises one or more polyphenols selected from the group consisting of hydroxytyrosol, tyrosol, and oleuropein. Preferably, the content of polyphenols in the olive extract is high, and more preferably the content of one or more polyphenols selected from the group consisting of hydroxytyrosol, tyrosol, and oleuropein is high.

The olive extract is commercially available, and the commercially available product may be used. Examples of the commercially available olive extracts may include “Olivex (trade name)” (GROUPE GRAP'SUD; France).

When the sleep quality improving agent of the present invention uses the olive extract as an active ingredient, the dosage of the sleep quality improving agent is not particularly limited so long as the effect of the present invention is not adversely affected, and may be suitably adjusted according to factors such as the age and the condition of the subject to be administered. The dosage per day for the administered subject to obtain the intended effect is generally 0.1 to 1000 mg, preferably 1 to 500 mg, more preferably 10 to 200 mg, and yet more preferably 20 to 180 mg as the dosage of the olive extract per day.

The sleep quality improving agent of the present invention may be formulated as it is and used as a final product such as food and drink products, pharmaceutical products, and quasi-pharmaceutical products. The sleep quality improving agent of the present invention may also be used as an additive for food and drink products, pharmaceutical products, and quasi-pharmaceutical products. This can impart the sleep quality improving effect to food and drink products, pharmaceutical products, and quasi-pharmaceutical products.

The present invention may comprise essentially the stilbene-based compound and/or the olive extract as active ingredients. That is, both the stilbene-based compound and the olive extract may be used simultaneously as the active ingredients. When the stilbene-based compound and the olive extract are used as the active ingredients, the sleep quality improving agent may be referred to as a sleep quality improving composition. The stilbene-based compound as the active ingredient may be a combination of two or more of stilbene-based compounds different in structure and the like. The olive extract as the active ingredient may be a combination of two or more of the olive extracts obtained by different extraction methods and the like.

The sleep quality improving agent of the present invention may comprise an ingredient other than active ingredients. One example of the ingredient other than the active ingredients is the one mainly ensuring the stability of the agent in storage and distribution such as storage stabilizer. In addition, the agent may include in advance one or two or more (preferably about one to three, and more preferably about one) ingredients selected from various ingredients constituting the intended final products such as food and drink products, pharmaceutical products, and quasi-pharmaceutical products.

The sleep quality improving agent of the present invention may be provided as a sleep quality improving composition by combining with the ingredients other than the stilbene-based compound and the olive extract.

Ingredients other than the stilbene-based compound and the olive extract included in the sleep quality improving composition of the present invention are not particularly limited so long as the object of the present invention is not adversely affected. Examples thereof may include pharmacologically acceptable additives such as excipients, disintegrants, binding agents, lubricants, coating agents, coloring agents, color formers, taste-masking agents, flavoring agents, antioxidants, antiseptics, tasting agents, acidulants, sweeteners, fortifiers, vitamin compounds, inflating agents, thickeners, and surfactants. One or two or more may be chosen from them, which do not adversely affect the sleep improving effect or various properties required for formulation such as formulation stability, and are suitable for the dosage form of final products. Further, the ingredients other than the stilbene-based compound and the olive extract may be one having the sleep improving effect besides the stilbene-based compound and the olive extract.

The dosage of the sleep quality improving composition of the present invention may be adjusted in the range of the amount of the stilbene-based compound and/or the olive extract as described above regarding the dosage of the sleep quality improving agent of the present invention.

The sleep quality improving composition of the present invention may be used in unchanged form as a final product such as food and drink products, pharmaceutical products, and quasi-pharmaceutical products. The composition may also be used as an additive for food and drink products, an additive for pharmaceutical products, and an additive for quasi-pharmaceutical products. This can impart the sleep quality improving effect to food and drink products, pharmaceutical products, and quasi-pharmaceutical products.

An administration form of the sleep quality improving agent and the sleep quality improving composition of the present invention is not particularly limited. Examples of the administration form may include oral administration such as buccal administration, and sublingual administration, and parenteral administration such as intravenous administration, intramuscular administration, subcutaneous administration, transdermal administration, transnasal administration, and pulmonary administration. Among them, the administration form that is minimally invasive is preferable, and the oral administration is more preferable.

A dosage form of the sleep quality improving agent and the sleep quality improving composition of the present invention is not particularly limited. The dosage form may be suitably determined depending on the selection from food and drink products, pharmaceutical products, and quasi-pharmaceutical products, and is not particularly limited. In case of oral administration, examples of the dosage form may include a liquid form (liquid agents), a syrup form (syrup agents), a solid form (tablets), a capsule form (capsules), a powder form (granules, fine particles), a soft capsule form (soft capsules), a semi-liquid form, a cream form, and a paste form. In case of parenteral administration, examples of the dosage form may include liquid agents (injections, nasal drops), and a mist form (sprays, inhalants).

An administration period of the sleep quality improving agent and the sleep quality improving composition of the present invention is not particularly limited, but they are generally administrated before bedtime, preferably between three hours before bedtime and bedtime, more preferably between two hours before bedtime and bedtime, further preferably between one hour before bedtime and bedtime, and particularly preferably one hour before bedtime.

The stilbene-based compound and/or the olive extract serving as active ingredients in the present invention have a remarkable sleep quality improving effect.

A term, “quality of sleep” in the present invention means that the exhausted body and brain can come to rest at a sleep. Fatigue cannot be fully relieved by sleeping for a long time without the quality of sleep. Examples of indexes measuring the quality of sleep may include the time from the onset of sleep to the appearance of the non-REM sleep (hereinafter referred to as sleep onset latency), the depth of the non-REM sleep in the initial stage of sleep, the ratio of the non-REM sleep time to the whole sleep time, less sleepiness on awakening, the sleep initiation as well as soundness of sleep, dreaming, a satisfaction with the length of sleep time, a fatigue feeling, and the depth of the non-REM sleep during the sleep onset latency and in the initial stage of sleep. Among them, the ratio of the non-REM sleep time to the whole sleep time, the less sleepiness feeling on awakening, the sleep initiation as well as the soundness of sleep, dreaming, the fatigue feeling, and the depth of the non-REM sleep in the initial stage of sleep are preferable. The non-REM sleep in the initial stage of sleep means the non-REM sleep that appears immediately after the onset of sleep until the appearance of the REM sleep. The depth of sleep becomes deeper as a delta wave power value increases. As shown in the Examples, the ratio of the non-REM sleep time to the whole sleep time may be confirmed by an electroencephalographic recording obtained by measuring a brain wave during sleep. The less sleepiness on awakening, the sleep initiation as well as the soundness of sleep, dreaming, the satisfaction with the length of sleep time, and the fatigue feeling may be evaluated by using the OSA sleep inventory MA version (Yamamoto Yukari, Tanaka Hideki, Takase Miki, Yamazaki Katsuo, Azumi Kazuo, and Shirakawa Shuichiro. Standardization of OSA sleep inventory for middle-aged and aged (MA version). Brain Science and Mental Disorders, 10, 401-409, 1999) as shown in the Examples. As shown in the Examples, the fatigue feeling may be evaluated by using a test paper sheet of visual analogue scales (VAS), which is recommended by Japanese Society of Fatigue Science and shown in the guideline on the method of evaluating the fatigue in anti-fatigue clinical evaluation of foods for specified health uses by Japanese Society of Fatigue Science. The sleep onset latency may be confirmed by electroencephalographic recording obtained by measuring brain waves during sleep. The depth of the non-REM sleep in the initial stage of sleep may be confirmed by delta wave power value of brain waves during sleep.

In the present invention, the wording “improving the quality of sleep” means that the quality of sleep described above is improved or increased. It may be judged whether the quality of sleep is improved, for example, by the fact that the ratio of the non-REM sleep time to the whole sleep time is increased, the fact that the less sleepiness feeling on awakening is improved, the fact that the sleep initiation as well as the soundness of sleep are improved, the fact that dreaming is improved (for example, no longer having dreams frequently or a nightmare), the fact that a recovery feeling from fatigue is increased (reduction of fatigue feeling), the fact that the sleep onset latency is reduced, and the fact that the non-REM sleep in the initial stage of sleep is deep. Whether the quality of sleep is improved may be confirmed by the conditions in the Examples.

According to the present invention, the quality of sleep can be improved effectively. Further, since the stilbene-based compound and the olive extract are material originally eaten as food and drink products, the sleep quality improving agent of the present invention comprising them as the active ingredients is safe for a living body and can be used by users without anxiety. Furthermore, as shown in the Examples below, the sleep quality improving agent of the present invention can reduce time of arousal during sleep and maintain sleep, and thereby can exhibit a particularly remarkable effect in recovery from fatigue. Thus, it is expected that the agent is applied to prevention and treatment of diseases associated with reduced quality of sleep such as diabetes, heart disease, hypertension, hyperlipidemia, and Alzheimer's disease. Therefore the sleep quality improving agent of the present invention is useful as final products such as food and drink products, pharmaceutical products, and quasi-pharmaceutical products.

A subject person to take the sleep quality improving agent or the sleep quality improving composition of the present invention is not particularly limited. Examples of the subject persons may include those who feel having a light sleep, those who feel sleepiness on awakening, those who feel difficulty in falling asleep, those who cannot sleep soundly (unable to sleep deeply), those who have bad dreams, those who feel fatigue, and those who does not recover from fatigue even after sleep. In addition, subject persons who doesn't have any substantial problem may also take the sleep quality improving agent or the sleep quality improving composition of the present invention routinely with an aim of improving and maintaining the quality of sleep.

Preferably, the sleep quality improving agent of the present invention is utilized as various types of food and drink products. Examples thereof may include beverages such as soft drinks, carbonated drinks, energy drinks, powdered drinks, fruit juices, milk-based drinks, and jelly drinks, confectionery such as cookies, cakes, chewing gums, candies, tablets, gummies, manju [steamed buns with filling], yokan [adzuki bean jellies], puddings, jellies, ice creams, and sherbets, processed marine products such as kamaboko [boiled fish paste], chikuwa [baked tubular rolls of fish pastes], and hanpen [boiled cakes made of ground fish], processed livestock products such as hamburgers, hams, sausages, wiener sausages, cheeses, butters, yogurt, fresh cream, and margarine, soups such as powdered soups and liquid soups, staple foods such as rices, noodles (dry noodles, raw noodles), breads, and cereal, and seasonings such as mayonnaise, shortening, dressing, sauce, tare [mop sauce], and soy sauce. The sleep quality improving agent of the present invention may be utilized as food and drink products such as health foods, functional foods, nutritional supplementary foods (supplements), foods for specified health use, foods for medical use, foods for the sick, baby foods, foods for nursing care, and foods for the elderly. Among them, it is preferably utilized as a health food and a functional food, and more preferably as a health food.

### EXAMPLES

The present invention is described below with reference to the Examples.

### 1-1. Evaluation of Sleep-Inducing Effect

### Examples 1 to 3 and Comparative Examples 1 to 3

### Measurement of Amount of Activity of Mice

In order to confirm the sleep-inducing effect of each of a grape extract, a melinjo extract, and an olive extract, the following tests were performed using each of the samples shown in Tables 1 to 3. Both the grape extract and the melinjo extract comprise the stilbene-based compounds and the multimers thereof.

The grape extract (trade name “VINEATROL20M,” manufactured by ACTICHEM S.A.) was suspended in a 0.5% aqueous solution of methylcellulose to prepare a sample solution of Example 1. The melinjo extract (trade name “MelinjoResveratrol-20,” manufactured by Luna YBF Inc., Ltd.) was suspended in a 0.5% aqueous solution of methylcellulose to prepare a sample solution of Example 2. The olive extract (trade name “Olivex HT6,” manufactured by La Gardonnenque S.C.A.) was suspended in a 0.5% aqueous solution of methylcellulose to prepare a sample solution of Example 3. A 0.5% aqueous solution of methylcellulose was used as a control solution for each sample solution (Comparative Examples 1, 2, and 3). In each Example and each Prescription Example of the present description, the commercially available products mentioned above are also used as the grape extract, the melinjo extract, and the olive extract, respectively.

If a sample has a function to induce sleep, administration of the sample to a subject always reduces its amount of activity. Therefore, the sample was evaluated for the sleep-inducing effect by confirming whether or not administering the sample solution to mice reduces their amount of activity.

Male C57BL/6 mice at 8 or 9 weeks of age were purchased from Japan SLC, Inc., and acclimated for 3 to 6 days. Mice were arranged into administration groups according to their amount of activity during acclimation. Just before the onset of the dark period, either the sample solution or the control solution was orally administrated in an amount of 10 mL/kg of the body weight, and then the amount of activity was measured for 24 hours after administration. There were 6 mice in each administration group (n=6).

The amount of activity was measured under the following conditions. The mice were individually housed in the cages, and infrared cameras were installed above them. The photographing range of infrared cameras is divided into 64 sections, so as to measure the number of times by which mice cross the section. The number of incidents was counted every 30 minutes.

TABLE 1 shows the type of samples, the oral administered amount of sample solutions, and the cumulative amount of activity during 5 hours after administration (times/5 hours) in Example 1 and Comparative Example 1. TABLE 2 shows the type of samples, the oral administered amount of sample solutions, and the cumulative amount of activity during 6 hours after administration (times/6 hours) in Example 2 and Comparative Example 2. TABLE 3 shows the type of samples, the oral administered amount of sample solutions, and the cumulative amount of activity during 8 hours after administration (times/8 hours) in Example 3 and Comparative Example 3.

TABLE 1
(SLEEP-INDUCING EFFECT ON MICE BY GRAPE EXTRACT)

| | | COMPARATIVE |
|---|---|---|
| | EXAMPLE 1 | EXAMPLE 1 |
| | | CONTROL |
| | | SOLUTION |
| | | (0.5% METHYL- |
| | GRAPE | CELLULOSE |
| | EXTRACT | SOLUTION) |
| ORAL ADMINISERED | 3000 | — |
| AMOUNT: mg/kg | | |
| (STILBENE-BASED | (600) | (0) |
| COMPOUND: mg/kg) | | |
| CUMULATIVE AMOUNT | 4262 | 7042 |
| OF ACTIVITY | | |
| DURING 5 HOURS AFTER | | |
| ADMINISTRATION | | |
| (TIMES/5 HOURS) | | |

TABLE 2
(SLEEP-INDUCING EFFECT ON MICE BY MELINJO EXTRACT)

| | EXAMPLE | COMPARATIVE |
|---|---|---|
| | 2 | EXAMPLE 2 |
| | MELINJO | CONTROL SOLUTION |
| | EXTRACT | (0.5% METHYL-CELLU- |
| | | LOSE SOLUTION) |
| ORAL ADMINISERED | 3000 | — |
| AMOUNT: mg/kg | | |
| (STILBENE-BASED | (600) | (0) |
| COMPOUND: mg/kg) | | |
| CUMULATIVE AMOUNT | 4733 | 9582 |
| OF ACTIVITY DURING | | |
| 6 HOURS AFTER ADMINI- | | |
| STRATION (TIMES/ | | |
| 6 HOURS) | | |

TABLE 3
(SLEEP-INDUCING EFFECT ON MICE BY OLIVE EXTRACT)

| | | COMPARATIVE |
|---|---|---|
| | EXAMPLE 3 | EXAMPLE 3 |
| | | CONTROL |
| | | SOLUTION |
| | | (0.5% METHYL- |
| | OLIVE | CELLULOSE |
| | EXTRACT | SOLUTION) |
| ORAL ADMINISERED | 3000 | — |
| AMOUNT: mg/kg | | |
| (TOTAL POLYPHENOL: mg/kg) | (900) | (0) |
| CUMULATIVE AMOUNT | 6048 | 10655 |
| OF ACTIVITY | | |
| DURING 8 HOURS AFTER | | |
| ADMINISTRATION | | |
| (TIMES/8 HOURS) | | |

It is clear from TABLE 1 that the cumulative amount of activity during 5 hours after administration in Example 1 using the grape extract as a sample was significantly decreased, as compared with Comparative Example 1 where the control solution was administered.

It is clear from TABLE 2 that the cumulative amount of activity during 6 hours after administration in Example 2 using the melinjo extract as a sample was significantly decreased, as compared with Comparative Example 2 where the control solution was administered.

It is clear from TABLE 3 that the cumulative amount of activity during 8 hours after administration in Example 3 using the olive extract as a sample was significantly decreased, as compared with Comparative Example 3 where the control solution was administered.

These results show that the grape extract, the melinjo extract, and the olive extract exert the sleep-inducing effect.

### 1-2. Evaluation of Sleep Quality Improving Effect 1

### Examples 4 to 6 and Comparative Examples 4 to 6

### Measurement of Brain Waves in Mice

In order to confirm the effect on sleep quality conferred by each of the grape extract, the melinjo extract, and the olive extract, the following tests were performed to measure brain waves.

Male C57BL/6 mice at 8 weeks of age purchased from Japan SLC Inc. were equipped with electrodes for an electroencephalograph and an electromyogram. After electrodes were equipped, mice were allowed to recover for 10 days in a recovery chamber. Then, mice were transferred to a recording chamber and connected with cables. After confirming whether or not brain waves are discernible by a brain wave analysis software, SleepSign (registered trademark) Ver. 3.0 (KISSEI COMTEC CO., LTD.), mice were acclimated for three days. Then, mice were measured for brain waves for 24 hours, so as to confirm whether the sleep-wakefulness rhythm was maintained, and arranged into administration groups. There were 6 mice in each administration group (n=6). Just before the onset of the dark period, 3 g/kg of the grape extract (Example 4), 3 g/kg of the melinjo extract (Example 5), 3 g/kg of the olive extract (Example 6), or 10 mL/kg of the control solution (Comparative Examples 4, 5, and 6) was each administered orally, and brain waves were measured for 24 hours. The control solution is a 0.5% by mass aqueous solution of methylcellulose. After recording, an automatic analysis for brain waves was performed with SleepSign (registered trademark) Ver. 3.0, then an evaluation practitioner confirmed the results of the automatic analysis, categorized sleep into three sleep stages: a wakefulness stage, a non-REM sleep stage, and a REM sleep stage. Then the time of each stage was calculated (TABLES 4 to 6).

TABLE 4
(SLEEP QUALITY IMPROVING
EFFECT ON MICE BY GRAPE EXTRACT)

| | | COMPERATIVE |
|---|---|---|
| | EXAMPLE 4 | EXAMPLE 4 |
| TIME OF SLEEP | | |
| STAGE DURING | | CONTROL |
| 12 HOURS AFTER | | SOLUTION (0.5% |
| ADMINISTRATION | GRAPE | METHYLCELLULOSE |
| (MIN.) | EXTRACT | SOLUTION) |
| WAKE | 322.9 | 472.2 |
| NON-REM SLEEP | 366.7 | 233.1 |

TABLE 5
(SLEEP QUALITY IMPROVING
EFFECT ON MICE BY MELINJO EXTRACT)

| | | COMPERATIVE |
|---|---|---|
| | EXAMPLE 5 | EXAMPLE 5 |
| TIME OF SLEEP | | |
| STAGE DURING 4 | | CONTROL |
| HOURS AFTER | | SOLUTION (0.5% |
| ADMINISTRATION | MELINJO | METHYLCELLULOSE |
| (MIN.) | EXTRACT | SOLUTION) |
| WAKE | 115.8 | 179.0 |
| NON-REM SLEEP | 117.7 | 57.8 |

TABLE 6
(SLEEP QUALITY IMPROVING
EFFECT ON MICE BY OLIVE EXTRACT)

| | | COMPERATIVE |
|---|---|---|
| | EXAMPLE 6 | EXAMPLE 6 |
| TIME OF SLEEP STAGE | | |
| DURING 6 HOURS AFTER | | CONTROL SOLUTION |
| ADMINISTRATION | OLIVE | (0.5% METHYLCELLULOSE |
| (MIN.) | EXTRACT | SOLUTION) |
| WAKE | 208.6 | 250.1 |
| NON-REM SLEEP | 145.3 | 103.6 |

It is clear from TABLE 4 that the time of wake was significantly decreased and the time of non-REM sleep was significantly increased in Example 4 using the grape extract as a sample, as compared with Comparative Example 4 where the control solution was administered.

It is clear from TABLE 5 that the time of wake was significantly decreased and the time of non-REM sleep was significantly increased in Example 5 using the melinjo extract as a sample, as compared with Comparative Example 5 where the control solution was administered.

It is clear from TABLE 6 that the time of wake was significantly decreased and the time of non-REM sleep was significantly increased in Example 6 using the olive extract as a sample, as compared with Comparative Example 6 where the control solution was administered.

The ratio of the non-REM sleep time to the total sleep time increases by administering the grape extract, the melinjo extract, or the olive extract, and thus these results show that the quality of sleep is improved. The results also show that these extracts can reduce the arousal and maintain the sleeping time.

### 1-3. Evaluation of Sleep Quality Improving Effect 2

### Examples 7 to 9 and Comparative Examples 7 to 9

### Measurement of Brain Waves in Human, and Survey of Feelings about Sleep

In order to confirm an effect on sleep quality in human conferred by the grape extract, the melinjo extract, and the olive extract, the following tests were performed using each sample shown in Tables 7 to 9.

Capsules formed by formulating the grape extract (content of stilbene-based compounds and multimers thereof per capsule: 14 mg) were prepared as a sample of Example 7. Capsules formed by formulating the melinjo extract (content of stilbene-based compounds and multimers thereof per capsule: 14 mg) were prepared as a sample of Example 8. Capsules formed by formulating the olive extract (content of polyphenols per capsule: 30 mg) were prepared as a sample of Example 9. As each sample of Comparative Examples 7 to 9, placebo capsules were prepared. Details of each composition are shown in TABLEs 7 to 9.

TABLE 7
(COMPOSITION OF CAPSULE AGENT COMPRISING GRAPE EXTRACT)

| INGREDIENTS (SINGLE ADMINISTERED AMOUNT: | EXAMPLE | COMPARATIVE |
|---|---|---|
| INGREDIENT AMOUNT PER CAPSULE, mg) | 7 | EXAMPLE 7 |
| GRAPE EXTRACT (mg) | 70 | 0 |
| (CONTENT OF STILBENE-BASED COMPOUNDS | (14) | |
| AND MULTIMERS THEREOF; mg) | | |
| CRYSTALLINE CELLULOSE (mg) | 180 | 250 |

TABLE 8
(COMPOSITION OF CAPSULE AGENT COMPRISING MELINJO EXTRACT)

| INGREDIENTS (SINGLE ADMINISTERED AMOUNT: | EXAMPLE | COMPARATIVE |
|---|---|---|
| INGREDIENT AMOUNT PER CAPSULE, mg) | 8 | EXAMPLE 8 |
| MELINJO EXTRACT (mg) | 70 | 0 |
| (CONTENT OF STILBENE-BASED COMPOUNDS | (14) | |
| AND MULTIMERS THEREOF; mg) | | |
| CRYSTALLINE CELLULOSE (mg) | 180 | 250 |

TABLE 9
(COMPOSITION OF CAPSULE AGENT COMPRISING OLIVE EXTRACT)

| INGREDIENTS (SINGLE ADMINISTERED AMOUNT: | EXAMPLE | COMPARATIVE |
|---|---|---|
| INGREDIENT AMOUNT PER CAPSULE, mg) | 9 | EXAMPLE 9 |
| OLIVE EXTRACT (mg) | 100 | 0 |
| (CONTENT OF POLYPHENOLS; mg) | (30) | |
| CRYSTALLINE CELLULOSE (mg) | 150 | 250 |

A questionnaire was carried out according to the Pittsburgh Sleep Quality Index in advance, and six adult men and women with relatively poor sleep state were selected as panelists. These six panelists were asked to take orally the sample of Example 7 one hour before going to bed, and be equipped with a two-electrode type portable electroencephalographic apparatus method, and then the brain waves were measured during sleep. Similarly, the same six panelists were asked to take orally the sample of Comparative Example 7 one hour before going to bed, and be equipped with the two-electrode type portable electroencephalographic apparatus, and then the brain waves were measured during sleep. In the next morning, the panelists were asked to answered a survey of feelings about sleep and a VAS test sheet for feeling of fatigue. The administration of each sample and the measurement were each carried out four times for each panelist.

The measurement was performed similarly as described above, except that the sample of Example 7 was replaced with the sample of Example 8 and the sample of Comparative Example 7 was replaced with the sample of Comparative Example 8. The measurement was performed similarly as described above, except that the sample of Example 7 was replaced with the sample of Example 9 and the sample of Comparative Example 7 was replaced with the sample of Comparative Example 9.

The average sleep time of each panelist was 6.5 hours, and the difference of sleep time between the panelist whose sleep time was the shortest and the panelist whose sleep time was the longest was 2 hours, and the difference was not significant.

(Analysis of Electroencephalogram)

Analysis of electroencephalogram was performed by Sleep well, Co., Ltd. by our request. A depth of sleep may be obtained by the delta wave power value of brain waves during sleep, and the higher the delta wave power value is, the deeper the depth of sleep is. The non-REM sleep appears immediately after sleeping, and then the REM sleep appears. The delta wave power value during the non-REM sleep appearing immediately after the onset of sleep until the appearance of the REM sleep was considered as the delta wave power value in the initial stage of sleep.

The delta wave power value varies from one individual to another. Therefore, the delta wave power value was measured four times for each panelist, and the average value of four measurements was calculated, then the change rate of the delta wave power value (%) as compared with placebo ingestion was calculated for each panelist by the following formula (1). The average value of these change rates obtained from 6 panelists was calculated (TABLES 10 to 12).

Change rate of delta wave power value (%)={(Average value of delta wave power value after sample ingestion of each Example)/(Average value of delta wave power value after placebo ingestion)}×100−100 [Formula (1)]

(Survey of Feelings about Sleep)

A survey of feelings about sleep was carried out by using the OSA sleep inventory MA version [Yamamoto Yukari, Tanaka Hideki, Takase Miki, Yamazaki Katsuo, Azumi Kazuo, and Shirakawa Shuichiro. Standardization of OSA sleep inventory for middle-aged and aged (MA version). Brain Science and Mental Dsorders, 10, 401-409, 1999]. The OSA sleep inventory MA version is composed of 20 question items and is an evaluation method for evaluating factor I: sleepiness feeling on awakening (i.e. less sleepiness feeling on rising), factor II: initiation and maintenance of sleep (i.e. easiness to get to sleep as well as improvement in soundness of sleep), factor III: dreaming (i.e. having a good dream), factor IV: recovery from fatigue (i.e. lack of the feeling of fatigue), and factor V: length of sleep time (i.e. satisfaction with the length of sleep time).

A score for each factor was calculated, and then the average value of four times measurements from each ingested sample was calculated for each panelist. The change rate of the score for each factor (%) as compared with placebo ingestion was calculated for each panelist by the following formula (2). The average value of these change rates obtained from 6 panelists was calculated (TABLES 13 to 15).

Change rate of score for each factor (%)={(Average value after sample ingestion of each Example)/(Average value after placebo ingestion)}×100−100 [Formula (2)]

(Survey on Feeling of Fatigue)

Survey on feeling of fatigue was carried out by using a test paper sheet of Visual Analogue Scales (VAS), which is recommended by Japanese Society of Fatigue Science and shown in the guideline on the method of evaluating the fatigue in anti-fatigue clinical evaluation of foods for specified health uses by Japanese Society of Fatigue Science. That is, there is a horizontal straight line of a certain length, where the left and right ends of the line indicate “the best feeling with no fatigue at all” and “the worst feeling of fatigue without motivation to do anything,” respectively, and the panelists replied their feelings when they wake up in the morning by placing the “X” mark on this straight line with reference to the representation of feelings indicated in the left and right ends of the straight line.

Distance from the left end of the line to the “X” mark was measured, and it is evaluated that the smaller the value of distance is, the less the panelist felt fatigue. The average value of four times measurements of this value in each ingested sample was calculated for each panelist, and the change rate of fatigue feeling (%) as compared with placebo ingestion was calculated for each panelist by the following formula (3). The average value of these change rates obtained from 6 panelists was calculated (TABLES 16 to 18).

[Formula (3)]

Change rate of fatigue feeling (%)={(Average value after sample ingestion of each Example)/(Average value after placebo ingestion)}×100−100 (4)

(Results of Electroencephalogram Analysis)

TABLE 10
(ADMINISTRATION OF GRAPE EXTRACT:
AVERAGE VALUE OF CHANGE RATES OF 6 PANELISTS)

| RESULTS OF | | SAMPLE OF |
|---|---|---|
| ELECTROENCEPHALOGRAM | SAMPLE OF | COMPARATIVE |
| ANALYSIS | EXAMPLE 7 | EXAMPLE 7 |
| DELTA WAVE POWER VALUE IN | 12.4% | 0% |
| INITIAL STAGE OF SLEEP | | |

TABLE 11
(ADMINISTRATION OF MELINJO EXTRACT:
AVERAGE VALUE OF CHANGE RATES OF 6 PANELISTS)

| RESULTS OF | | SAMPLE OF |
|---|---|---|
| ELECTROENCEPHALOGRAM | SAMPLE OF | COMPARATIVE |
| ANALYSIS | EXAMPLE 8 | EXAMPLE 8 |
| DELTA WAVE POWER VALUE IN | 2.4% | 0% |
| INITIAL STAGE OF SLEEP | | |

TABLE 12
(ADMINISTRATION OF OLIVE EXTRACT:
AVERAGE VALUE OF CHANGE RATES OF 6 PANELISTS)

| RESULTS OF | | SAMPLE OF |
|---|---|---|
| ELECTROENCEPHALOGRAM | SAMPLE OF | COMPARATIVE |
| ANALYSIS | EXAMPLE 9 | EXAMPLE 9 |
| DELTA WAVE POWER VALUE IN | 6.7% | 0% |
| INITIAL STAGE OF SLEEP | | |

TABLE 10 shows that when the sample of Example 7 (capsules comprising the grape extract) was orally ingested, the delta wave power value in the initial stage of sleep was higher and the non-REM sleep became sufficiently deeper, as compared with oral ingestion of placebo (Comparative Example 7).

TABLE 11 shows that when the sample of Example 8 (capsules comprising the melinjo extract) was orally ingested, the delta wave power value in the initial stage of sleep was higher and the non-REM sleep became sufficiently deeper, as compared with oral ingestion of placebo (Comparative Example 8).

TABLE 12 shows that when the sample of Example 9 (capsules comprising the olive extract) was orally ingested, the delta wave power value in the initial stage of sleep was higher and the non-REM sleep became sufficiently deeper, as compared with oral ingestion of placebo (Comparative Example 9).

These results show that the non-REM sleep in the initial stage of sleep becomes deeper and the quality of sleep is effectively improved by the administration of the grape extract, the melinjo extract, or the olive extract.

(Results of Survey of Feelings about Sleep)

TABLE 13
(ADMINISTRATION OF GRAPE EXTRACT:
AVERAGE VALUE OF CHANGE RATES OF 6 PANELISTS)

| | | SAMPLE OF |
|---|---|---|
| RESULTS OF OSA SLEEP | SAMPLE OF | COMPARATIVE |
| INVENTORY MA VERSION | EXAMPLE 7 | EXAMPLE 7 |
| FACTOR I (SLEEPINESS FEELING | 39.3% | 0% |
| ON AWAKENING) | | |
| FACTOR II (INITIATION AND | 28.8% | 0% |
| MAINTENANCE OF SLEEP) | | |
| FACTOR III (DREAMING) | 6.1% | 0% |
| FACTOR IV (RECOVERY FROM | 40.8% | 0% |
| FATIGUE) | | |
| FACTOR V (LENGTH OF SLEEP | 9.1% | 0% |
| TIME) | | |

TABLE 14
(ADMINISTRATIONOF MELINJO EXTRACT:
AVERAGE VALUE OF CHANGE RATES OF 6 PANELISTS)

| | | SAMPLE OF |
|---|---|---|
| RESULTS OF OSA SLEEP | SAMPLE OF | COMPARATIVE |
| INVENTORY MA VERSION | EXAMPLE 8 | EXAMPLE 8 |
| FACTOR I (SLEEPINESS FEELING | 16.3% | 0% |
| ON AWAKENING) | | |
| FACTOR II (INITIATION AND | 20.0% | 0% |
| MAINTENANCE OF SLEEP) | | |
| FACTOR III (DREAMING) | 32.6% | 0% |
| FACTOR IV (RECOVERY FROM | | |
| FATIGUE) | 15.8% | 0% |
| FACTOR V (LENGTH OF SLEEP | | |
| TIME) | 1.0% | 0% |

TABLE 15
(ADMINISTRATION OF OLIVE EXTRACT:
AVERAGE VALUE OF CHANGE RATES OF 6 PANELISTS)

| | | SAMPLE OF |
|---|---|---|
| RESULTS OF OSA SLEEP | SAMPLE OF | COMPARATIVE |
| INVENTORY MA VERSION | EXAMPLE 9 | EXAMPLE 9 |
| FACTOR I (SLEEPINESS FEELING | 34.6% | 0% |
| ON AWAKENING) | | |
| FACTOR II (INITIATION AND | 25.6% | 0% |
| MAINTENANCE OF SLEEP) | | |
| FACTOR III (DREAMING) | 2.3% | 0% |
| FACTOR IV (RECOVERY FROM | 22.6% | 0% |
| FATIGUE) | | |
| FACTOR V (LENGTH OF SLEEP | 14.7% | 0% |
| TIME) | | |

TABLE 13 shows the following. When the sample of Example 7 (capsules comprising the grape extract) was orally ingested, each score of factor I (sleepiness feeling on awakening), factor II (initiation and maintenance of sleep), factor III (dreaming), factor IV (recovery from fatigue), and factor V (length of sleep time) was increased, and the change rates of the respective scores were all higher, and therefore improved, as compared with oral ingestion of placebo (Comparative Example 7).

TABLE 14 shows the following. When the sample of Example 8 (capsules comprising the melinjo extract) was orally ingested, each score of factor I (sleepiness feeling on awakening), factor II (initiation and maintenance of sleep), factor III (dreaming), factor IV (recovery from fatigue), and factor V (length of sleep time) was increased, and the change rates of the respective scores were all higher, and therefore improved, as compared with oral ingestion of placebo (Comparative Example 8).

TABLE 15 shows the following. When the sample of Example 9 (capsules comprising the olive extract) was orally ingested, each score of factor I (sleepiness feeling on awakening), factor II (initiation and maintenance of sleep), factor III (dreaming), factor IV (recovery from fatigue), and factor V (length of sleep time) was increased, and the change rates of the respective scores were all higher, therefore improved, as compared with oral ingestion of placebo (Comparative Example 9).

These results show that the sleep quality improving effect was exerted by administering the grape extract, the melinjo extract, or the olive extract, and thus remained sleepiness feeling on awakening was improved. The results also show that the sleep quality improving effect was exerted, and thus the sleep initiation and the improvement in soundness of sleep were improved. The results also show that the sleep quality improving effect was exerted, and thus the condition of dreaming was improved. The results also show that the sleep quality improving effect was exerted, and thus the recovery feeling from fatigue was improved. Further, the results show that the sleep quality improving effect was exerted, and thus the satisfaction with the length of sleep time was improved.

(Results of Survey on Feeling of Fatigue)

TABLE16
(ADMINISTRATION OF GRAPE EXTRACT:
AVERAGE VALUE OF CHANGE RATES OF 6 PANELISTS)

| RESULTS OF VAS | SAMPLE OF | SAMPLE OF COMPARATIVE |
|---|---|---|
| TEST | EXAMPLE 7 | EXAMPLE 7 |
| FEELING OF | −11.2 % | 0 % |
| FATIGUE | | |

TABLE 17
(ADMINISTRATION OF MELINJO EXTRACT:
AVERAGE VALUE OF CHANGE RATES OF 6 PANELISTS)

| RESULTS OF VAS | SAMPLE OF | SAMPLE OF COMPARATIVE |
|---|---|---|
| TEST | EXAMPLE 8 | EXAMPLE 8 |
| FEELING OF | −9.4 % | 0 % |
| FATIGUE | | |

TABLE 18
(ADMINISTRATION OF OLIVE EXTRACT:
AVERAGE VALUE OF CHANGE RATES OF 6 PANELISTS)

| RESULTS OF VAS | SAMPLE OF | SAMPLE OF COMPARATIVE |
|---|---|---|
| TEST | EXAMPLE 9 | EXAMPLE 9 |
| FEELING OF | −4.0% | 0 % |
| FATIGUE | | |

TABLE 16 shows that when the sample of Example 7 (capsules comprising the grape extract) was orally ingested, the fatigue feeling was decreased, as compared with oral ingestion of the placebo in Comparative Example 7.

TABLE 17 shows that when the sample of Example 8 (capsules comprising the melinjo extract) was orally ingested, the fatigue feeling was decreased, as compared with oral ingestion of the placebo in Comparative Example 8.

TABLE 18 shows that when the sample of Example 9 (capsules comprising the olive extract) was orally ingested, the fatigue feeling was decreased, as compared with oral ingestion of the placebo in Comparative Example 9.

These results show that, the sleep quality improving effect was exerted by administering the grape extract, the melinjo extract, or the olive extract, and thus fatigue was no longer felt (fatigue feeling was reduced).

Prescription examples of the sleep quality improving agent and the sleep quality improving composition of the present invention are shown below.

### Prescription Example 1

### Tablets Formed by Comprising the Grape Extract or the Melinjo Extract

Tablets were produced, according to the standard method, by tableting 140 mg of a grape extract or a melinjo extract, 155 mg of crystalline cellulose, and 5 mg of sucrose fatty acid ester.

### Prescription Example 2

### Tablets Formed by Comprising the Olive Extract (1)

Tablets were produced, according to the standard method, by tableting 100 mg of an olive extract, 190 mg of crystalline cellulose, 5 mg of carboxymethylcellulose-Ca, and 5 mg of sucrose fatty acid ester.

### Prescription Example 3

### Tablets Formed by Comprising the Olive Extract (2)

According to a routine method, tablets were produced using the following materials:

108 mg of granulated products; 100 mg of an olive extract; 110 mg of sorbitol; 15 mg of partially pregelatinized modified starch; 75 mg of magnesium phosphate; 3 mg of calcium stearate; 40 mg of menthol particles; and 5 mg of aspartame.

The above mentioned granulated products were prepared by adding 4000 g of 6% by mass aqueous solution of hydroxypropyl cellulose to 1935 g of erythritol and 300 g of cornstarch. The average particle diameter of the granulated products was 290 μm.

### Prescription Example 4

### Soft Capsules Formed by Comprising the Grape Extract or the Melinjo Extract

The contents were produced by using the following materials:

140 mg of a grape extract or a melinjo extract; 110 mg of vegetable oil and fat; 10 mg of glycerine fatty acid ester; 10 mg of beeswax.

A according to the conventional method, soft capsules were produced by using the above mentioned contents and gelatin derived from swine.

(Prescription Example 5: Soft capsules formed by comprising the olive extract)

The contents were produced by using the following materials:

100 mg of an olive extract; 110 mg of vegetable oil and fat; 10 mg of glycerine fatty acid ester; 10 mg of beeswax.

According to the conventional method, soft capsules were produced by using the above mentioned contents and gelatin derived from swine according to a routine method.

1. A sleep quality improving composition comprising a stilbene-based compound and/or an olive extract as an active ingredient.

2. The sleep quality improving composition according to claim 1, wherein the composition comprises the stilbene-based compound in the form of a plant extract comprising the stilbene-based compound.

3. The sleep quality improving composition according to claim 2, wherein the composition comprises the stilbene-based compound, and the stilbene-based compound is in the form of a Vitaceae plant extract comprising the stilbene-based compound, or a Gnetaceae plant extract comprising the stilbene-based compound.

4. The sleep quality improving composition according to claim 1, wherein the composition comprises the olive extract in the form of an extract isolated from a fruit of olive.

5. The sleep quality improving composition according to claim 1, wherein the composition comprises the olive extract, and the olive extract in the form of an olive extract comprising one or more polyphenols selected from the group consisting of hydroxytyrosol, tyrosol, and oleuropein.

6. The sleep quality improving composition according to claim 1, wherein the composition increases a ratio of non-REM sleep time to a whole sleep time, deepens non-REM sleep at an initial stage of sleep, lessens a feeling of sleepiness upon awakening, improves sleep initiation and soundness of sleep, improves dreaming, improves a feeling of fatigue, and/or improves satisfaction with a length of sleep time.

7-12. (canceled)

13. The sleep quality improving composition according to claim 1, wherein the composition is administered orally.

14-16. (canceled)

17. A method of improving sleep quality of a human subject in need thereof, comprising administering to the human subject a sleep quality-improving amount of a composition comprising a stilbene-based compound and/or an olive extract.

18. The method according to claim 17, wherein the composition comprises the stilbene-based compound in the form of a plant extract comprising the stilbene-based compound.

19. The method according to claim 18, wherein the composition comprises the stilbene-based compound, and the stilbene-based compound is in the form of a Vitaceae plant extract comprising the stilbene-based compound, or a Gnetaceae plant extract comprising the stilbene-based compound.

20. The method according to claim 17, wherein the composition comprises the olive extract in the form of an extract isolated from a fruit of olive.

21. The method according to claim 17, wherein the composition comprises the olive extract, and the olive extract is in the form of an olive extract comprising one or more polyphenols selected from the group consisting of hydroxytyrosol, tyrosol, and oleuropein.

22. The method according to claim 17, wherein the composition increases a ratio a ratio of non-REM sleep time to a whole sleep time, deepens non-REM sleep at an initial stage of sleep, lessens a feeling of sleepiness upon awakening, improves sleep initiation and soundness of sleep, improves dreaming, improves a feeling of fatigue, and/or improves satisfaction with a length of sleep time.

23. The method according to claim 17, wherein the composition is administered orally.
